(19) United States
(12) Patent Application Publication
(10) Pub. No.: US 2007/0212404 A1
(43) Pub. Date: Sep. 13, 2007

(54) Liposome And Method Of Preparing The Same

(75) Inventors: Kimoon Kim, Pohang-city (KR); Hyung Kun Lee, Pohang-city (KR); Kyung Min Park, Pohang-city (KR); Young Jin Jeon, Pohang-city (KR); Dong-Hyun Oh, Pohang-city (KR); Dongwoo Kim, Pohang-city (KR)

Correspondence Address: ROTHWELL, FIGG, ERNST & MANBECK, P.C., 1425 K STREET, N.W., SUITE 800, WASHINGTON, DC, 20005, US

(73) Assignee: POSTECH ACADEMY-INDUSTRY FOUNDATION, Pohang-city, Kyungsangbuk-do (KR)

(21) Appl. No.: 11/587,098

(22) PCT Filed: Apr. 19, 2005
(86) PCT No.: PCT/KR05/01110

§ 371 Date: Oct. 20, 2006

(30) Foreign Application Priority Data
Apr. 21, 2004 (KR) 10-2004-0027577

(51) Int. Cl. A61K 9/127; A61K 38/21; A61K 38/19; A61K 39/395; A61K 38/28; A61K 38/26; A61K 38/24; A61K 38/48; C12N 15/88; A61K 48/00

(52) U.S. Cl. 424/450; 514/12; 514/393; 424/85.1; 424/133.1; 424/85.4; 424/94.63; 514/3; 514/282; 514/171; 514/420; 514/28; 514/44; 435/458

(57) ABSTRACT

Provided are a liposome formed by self-assembling a cucurbituril derivative and a method of preparing the liposome.

### CROSS REFERENCE TO RELATED APPLICATION

This application is a U.S.C. § 371 National Phase Entry Application from PCT/KR2005/001110, filed Apr. 19, 2005, and designating the U.S.

### BACKGROUND OF THE INVENTION

1. Field of the Invention

The present invention relates to a liposome and a method of preparing the same, and, more particularly to, a liposome composed of a cucurbituril derivative and a method of preparing the liposome.

2. Description of the Related Art

A large amount of capital and time has been invested into the development of pharmacologically active substances. An effective application of these pharmacologically active substances requires efficient drug delivery systems. Thus, vigorous research has been conducted on the development of drug delivery systems in many countries and gene delivery substances relating to these drug delivery systems are available, forming a very large market. The size of the future biotechnology-related market is expected to increase greatly the research and development of drug delivery systems in Korea must be pushed forward. An example of a newly developed and currently commercially available drug delivery system in Korea is a microemulsion formulation of cyclosporin (trade name: Implanta) used as an immunosuppressant, exported from Hanmi Pharmaceutical Co. (Korea) to Novartis. Another example is a transdermal absorbent Ketotop (Pacific Pharmaceutical Co., Korea). Based on the successful commercialization of developed drug delivery systems, vigorous research into drug delivery systems has been conducted by the departments of chemistry, chemical engineering, pharmacy, medicine, and the like of numerous domestic universities, and research institutes of pharmaceutical companies, governmental research institutes, and industrial chemistry-related research institutes. Furthermore, research has been conducted on various drug delivery systems for genes, proteins, and organic compounds, various administrative methods such as oral, transdermal, transnasal administration and injection, and drug delivery systems targeted to specific organs such as the brain, kidneys, liver, etc.

Conventional methods of effectively delivering pharmacologically active substances, for example, drugs and genes, include a method using a retroviral vector, a method using nanoparticles, a method using a liposome, etc.

The method using a retrovirus has a high transfection efficiency, but is limited in use it induces an in vivo immune reaction.

Artificially synthesized nanoparticles do not induce an in vivo immune reaction and are relatively stable, thus having lower production costs. However, the nanoparticles cannot effectively encapsulate drugs and genes.

A liposome designed as a drug delivery system refers to a vesicle that has the structure of a bimolecular layer and is obtained by suspending an amphiphile in water. A liposome can encapsulate a large amount of pharmacologically active substances. A liposome having a modified surface can be specifically transported to a target site, and thus, can be used as a targeting liposome which can increase the concentration of a pharmacologically active substance only around the target organ or a target tissue.

The following important requirements in the development of a drug delivery system must be satisfied: side effects must be decreased; a stable formulation between a drug and a drug delivery system must be formed to prevent loss and degeneration of the drug and to ensure stable drug delivery; and a drug must be stably delivered to a targeted organ or cell. Continuous development of various drug delivery systems satisfying the above requirements is required. Hitherto, however, there have not been many drug delivery systems which are excellent in terms of all of thermoplasticity, biocompatibility, biodegradability, productivity, processability, and the like. Therefore, a promising new drug delivery systems must be developed. Furthermore, active participation in the development of drug delivery systems, which is important for new drug development, is required to keep pace with worldwide studies about the development of various drug delivery systems. In order to play a leading role in the development of drug delivery systems, new candidate compounds for drug delivery systems must be discovered.

Cucurbituril was first reported by R. Behrend, E. Meyer, F. Rusche in 1905. In 1981, this substance was rediscovered by W. Mock and his coworkers. W. Mock and his coworkers correctly characterized cucurbituril as a hexameric macrocyclic compound with the chemical formula C₃₆H₃₆N₂₄O₁₂, which was confirmed by X-ray diffraction (J. Am. Chem. Soc. 1981, 103, 7367). They named it cucurbit[6]uril. Since then, an improved method of synthesizing cucurbit[6]uril has been disclosed (DE 196 03 377 A1).

In 2000, Kimoon Kim and his coworkers reported the improved preparation and separation of cucurbit[6]uril and its homologues, cucurbitu[n]rils (n=5, 7, 8), and identified their structures by X-ray diffraction (J. Am. Chem. Soc. 2000, 122, 540).

Meanwhile, WO 00/68232 discloses cucurbitu[n]ril represented by Reference Diagram 1 below:

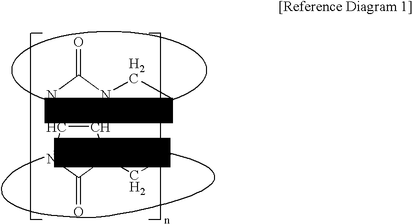

wherein n is an integer from 4 to 12.

The above-described cucurbituril derivatives are compounds including unsubstituted glycoluril monomer units.

Cucurbituril is a macrocyclic compound and has a lipophilic cavity and two hydrophilic entrances at upper and lower ends. Lipophilic interactions occur in the lipophilic cavity of the cucurbituril, and hydrogen bonding, polar-polar interactions, and positive charge-polar interactions occur in the two hydrophilic entrances, which each has six carbonyl groups. Therefore, cucurbituril can include various compounds by forming very stable non-covalent bonds with these compounds. Cucurbituril forms a complex, particularly with a compound having an amino group or a carboxyl group, by forming a very stable non-covalent linkage. Based on such characteristics, studies about the application of cucurbituril in various drug delivery systems have been continuously conducted.

Recently, the present inventors reported a complex formation between oxaliplatin approved as an anticancer agent by the Food and Drug Administration (FDA) and cucurbituril used as a drug delivery system via a stable non-covalent bond (PCT/KR02/01755). Furthermore, the present inventors reported a cucurbituril-containing pseudo-rotaxane with an enhanced DNA binding capacity and the use of a cucurbituril-based dendrimer as a gene delivery system (Angew. Chem. Int. Ed., 2000 and 2001). Also, the present inventors reported a pharmaceutical composition comprising nanoparticles and a pharmacologically active substance encapsulated in the nanoparticles and a method of preparing the same based on the non-covalent binding properties of cucurbiturils and the easy introduction of various functional groups into cucurbituril derivatives (Korean Patent Application No. 2003-0051841).

Therefore, under the necessity to develop new drug delivery systems, the present inventors conducted research on new drug delivery systems based on a cucurbituril derivative and discovered the present invention.

### SUMMARY OF THE INVENTION

The present invention provides a liposome composed of a cucurbituril derivative.

The present invention also provides a liposome composed of a cucurbituril derivative and modified with a targeting compound.

The present invention also provides a liposome composed of a cucurbituril derivative and encapsulating a pharmacologically active substance.

The present invention also provides a method of preparing the above liposome.

According to an aspect of the present invention, there is provided a liposome formed by self-assembling a cucurbituril derivative having formula 1:

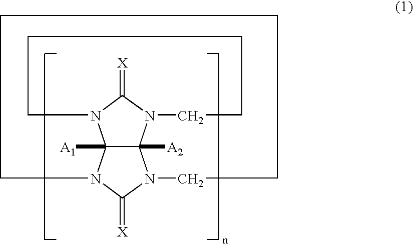

wherein

X is O, S, or NH,

A₁ and A₂ are respectively OR¹ and OR², SR¹ and SR², or NHR¹ and NHR²,

each of R¹ and R² is independently selected from the group consisting of a hydrogen atom, a substituted or unsubstituted C₁-C₃₀ alkyl, a substituted or unsubstituted C₂-C₃₀ alkenyl, a substituted or unsubstituted C₂-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylthiol, a substituted or unsubstituted C₁-C₃₀ alkoxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, and a substituted or unsubstituted C₄-C₂₀ heteroarylalkyl, and

n is an integer from 4 to 20.

A surface of the liposome may be modified by including a targeting compound in a cavity of the cucurbituril derivative composing the liposome such that a targeting moiety of the targeting compound is exposed to the outside of the liposome.

A pharmacologically active substance may be encapsulated as a guest molecule in the liposome or the liposome having its surface modified by the targeting compound.

According to another aspect of the present invention, there is provided a method of preparing a liposome formed by self-assembling the cucurbituril derivative having formula 1, the method comprising: dissolving a cucurbituril derivative having formula 1 in an organic solvent and drying the resultant solution; and adding water to the dried compound and dispersing the compound.

According to still another aspect of the present invention, there is provided a method of preparing a liposome in which a targeting compound is included in a cavity of the cucurbituril derivative having formula 1 composing the liposome, the method comprising: dissolving the cucurbituril derivative having formula 1 in an organic solvent and drying the resultant solution; adding water to the dried compound and dispersing the compound; adding a targeting compound or a solution of the targeting compound to the dispersion and stirring the resultant mixture; and removing a residual unembedded targeting compound by dialysis.

According to yet another aspect of the present invention, there is provided a method of preparing a liposome in which a pharmacologically active substance is encapsulated as a guest molecule, the method comprising: dissolving the cucurbituril derivative having formula 1 in an organic solvent and drying the resultant solution; adding an aqueous solution of the pharmacologically active substance to the dried compound and dispersing the compound; and removing a residual non-encapsulated pharmacologically active substance in the dispersion by dialysis.

According to a further aspect of the present invention, there is provided a method of preparing a liposome in which a pharmacologically active substance is encapsulated and a targeting compound is embedded in a surface of the liposome, the method comprising: dissolving a cucurbituril derivative having formula 1 in an organic solvent and drying the resultant solution; adding an aqueous solution of the pharmacologically active substance to the dried compound and dispersing the compound; adding a targeting compound or a solution of the targeting compound to the dispersion and stirring the resultant mixture; and removing a residual non-encapsulated pharmacologically active substance and a residual unembedded targeting compounds by dialysis.

### BRIEF DESCRIPTION OF THE DRAWINGS

The above and other features and advantages of the present invention will become more apparent by describing in detail exemplary embodiments thereof with reference to the attached drawings in which:

FIG. 1 is a transmission electron microscope (TEM) photo of a liposome formed by self-assembling {3-{2-[2-(2-methoxy-ethoxy)-ethoxy]-ethylsulfanyl}-propyloxy}12 cucurbituril; and

FIG. 2 is a schematic view of a pharmacologically active substance encapsulated liposome having a surface modified with a targeting compound having formula 2.

### DETAILED DESCRIPTION OF THE INVENTION

Hereinafter, the present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art.

In an embodiment of the present invention, a liposome is formed by self-assembling a cucurbituril derivative. The liposome includes a space filled with an aqueous solution and has a diameter of several tens to 1000 nm. The cucurbituril derivative composing the liposome has formula 1:

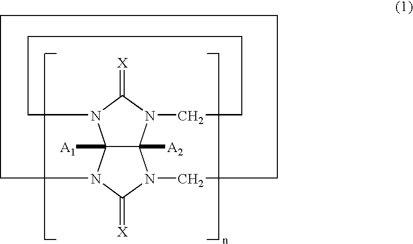

wherein

X is O, S, or NH,

A₁ and A₂ are respectively OR¹ and OR², SR¹ and SR², or NHR¹ and NHR²,

each of R¹ and R² is independently selected from the group consisting of a hydrogen atom, a substituted or unsubstituted C₁-C₃₀ alkyl, a substituted or unsubstituted C₁-C₃₀ alkenyl, a substituted or unsubstituted C₁-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylthiol, a substituted or unsubstituted C₁-C₃₀ alkoxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, and a substituted or unsubstituted C₄-C₂₀ heteroarylalkyl, and

n is an integer from 4 to 20.

The liposome formed by self-assembling the cucurbituril derivative having formula 1 may be provided with a targeting property by modifying its surface with a targeting compound. The cucurbituril derivative having formula 1 is an inclusion compound which has a cavity in its molecule, as illustrated in the Reference Diagram 1 above, and thus a targeting compound can be included in the cavity.

Examples of the targeting compound that can be included in the cavity of the cucurbituril on a surface of the liposome include, but are not limited to, a compound having formula 2:
A-B-T (2)

wherein

A is 1,3-diaminopropyl, 1,4-diaminobutyl, 1,5-diaminopentyl, 1,6-diaminohexyl, sperminyl, spermidinyl, propylamino, butylamino, pentylamino, hexylamino, biologinyl, pyridinyl, ferrocenyl, or amino acid,

B is a hydrogen atom, a substituted or unsubstituted C₁-C₃₀ alkyl, a substituted or unsubstituted C₁-C₃₀ alkenyl, a substituted or unsubstituted C₁-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylsulfanyl, a substituted or unsubstituted C₁-C₃₀ alkyloxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, or a substituted or unsubstituted C₄-C₂₀ heteroarylalkyl, and

T is a targeting moiety selected from the group consisting of a saccharide, a polypeptide, a protein, and a gene.

In the compound having formula 2, examples of the saccharide for T may include, but not limited to, glucose, mannose, and galactose.

In the compound having formula 2, examples of the protein for T may include, but not limited to, lectin, selectin, and transferrin.

A structure in which the targeting compound having formula 2 is included in the hole of the cucurbituril in the surface of the liposome is illustrated in Reference Diagram 2 below:

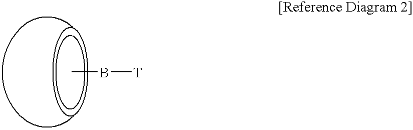

In the compound having formula 2, A is designed to be easily included in the cucurbituril derivative exposed on the surface of the liposome when the cucurbituril derivative forms the liposome. Due to this strategy, as illustrated in Reference Diagram 2, the surface of the liposome can be modified with the targeting moiety T, which is connected to A via a linkage portion B.

A liposome formed by self-assembling the cucurbituril derivative and a liposome embedding the targeting compound in the surface thereof can function as drug carriers. Thus, a pharmacologically active substance can be encapsulated as a guest molecule into a hole of the liposome. Especially, the drug encapsulated in the liposome embedding the targeting compound specifically reacts with a target site in the body, and thus a side effect due to the reaction of the drug with non-targeted sites can be prevented.

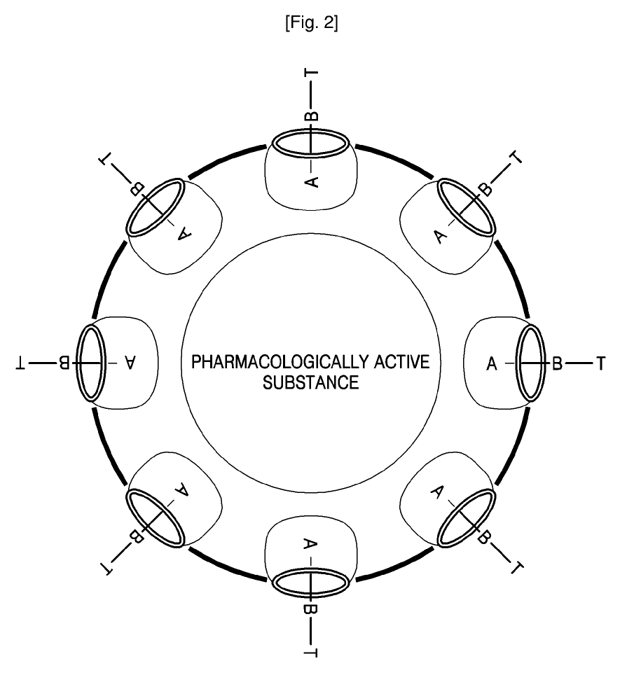

FIG. 2 is a schematic view of a pharmacologically active substance encapsulated liposome having a surface modified with the targeting compound having formula 2.

Examples of the pharmacologically active substance may include an organic compound, a protein, and a gene, etc.

Examples of the organic compound may include, but are not limited to, hydrocortisone, prednisolone, spironolactone, testosterone, megesterol acetate, danasole, progesterone, indomethacin, amphotericin B, and a mixture thereof.

Examples of the protein may include, but are not limited to, a human growth hormone, a G-CSF (granulocyte colony-stimulating factor), GM-CSF granulocyte-macrophage colony-stimulating factor), erythropoietin, a vaccine, an antibody, insulin, glucagon, calcitonin, an ACTH (adrenocorticotropic hormone), somatostatin, somatotropin, somatomedin, parathyroid hormone, thyroid hormone, a hypothalamus secretion, prolactin, endorphin, a VEGF (vascular endothelial growth factor), enkephalin, vasopressin, a nerve growth factor, non-naturally occurring opioid, interferon, asparaginase, alginase, superoxide dismutase, trypsin, chymotrypsin, pepsin, and a mixture thereof.

A method of preparing a liposome by self-assembling the cucurbituril derivative having formula 1, includes: dissolving the cucurbituril derivative having formula 1 in an organic solvent and drying the resultant solution; and adding water to the dried compound and dispersing the compound.

A method of preparing a pharmacologically active substance encapsulated liposome includes: dissolving the cucurbituril derivative having formula 1 in an organic solvent and drying the resultant solution; adding an aqueous solution of the pharmacologically active substance to the dried compound and dispersing the compound; and removing a residual non-encapsulated pharmacologically active substance in the dispersion by dialysis.

A method of preparing a liposome embedding a targeting compound includes: dissolving the cucurbituril derivative having formula 1 in an organic solvent and drying the resultant solution; adding water to the dried compound and dispersing the compound; adding the targeting compound or a solution of the targeting compound to the dispersion and stirring the resultant mixture; and removing a residual non-encapsulated targeting compound by dialysis.

A method of preparing a liposome in which a pharmacologically active substance is encapsulated and a targeting compound is embedded: dissolving a cucurbituril derivative having formula 1 in an organic solvent and drying the resultant solution; adding an aqueous solution of a pharmacologically active substance to the dried compound and dispersing the compound; adding the targeting compound or a solution of the targeting compound to the dispersion and stirring the resultant mixture; and removing a residual non-encapsulated pharmacologically active substance and a residual non-encapsulated targeting compound by dialysis.

In all of the above-described methods of preparing a liposome, the organic solvent may be a solvent capable of solubilizing the cucurbituril derivative. Examples of the organic solvent may include, but are not limited to, chloroform, methanol, dimethylsulfoxide, dichloromethane, dimethylformamide, tetrahydrofuran, and a mixture thereof.

In the adding of water or the aqueous solution of the pharmacologically active substance to the dried cucurbituril derivative, a volume of the added water or aqueous solution may be varied such that a concentration of the cucurbituril derivative lies in a range of 10⁻⁴ to 10⁻² M. If the concentration of the cucurbituril derivative is less than 10⁻⁴ M or greater than 10⁻² M, the liposome cannot be easily formed. After the addition of the water, the cucurbituril derivative must be uniformly dispersed in the water, preferably, by sonication with a sonicator. The dispersing may be performed at any temperature at which a liposome can be formed, preferably at 10-60° C.

To form a liposome with a modified surface property by embedding a compound of formula 2 in the surface of liposome or the pharmacologically active substance encapsulated liposome, the solution of the targeting compound is added to the dispersion of the liposome, and then the resultant mixture is dispersed. This dispersing process may be performed at a temperature ranging from room temperature to 60° C. If the stirring temperature is set too high, the solvent evaporates, thereby resulting in a modification or decomposition of the liposome. Alternatively, the targeting compound may be directly added to the dispersion of the liposome instead of dissolving the targeting compound in a solvent prior to the adding to the dispersion of the dispersion.

As described above, a liposome or a pharmacologically active substance encapsulated liposome may be formed by self-assembling the cucurbituril derivative in water or a aqueous solution of the pharmacologically active substance and dispersing the same therein. Further, a liposome having a modified surface property due to a targeting compound embedded therein may be prepared by embedding the targeting compound having formula 2 therein. The liposomes may have diameters of several tens to 1000 nm and can be observed using an optical microscope, light-scattering, a scanning electron microscope (SEM), or a transmission electron microscope (TEM).

Hereinafter, the present invention will be described in more detail with reference to the following examples. The following examples are given for illustrative purposes and are not intended to limit the scope of the invention.

### EXAMPLE 1

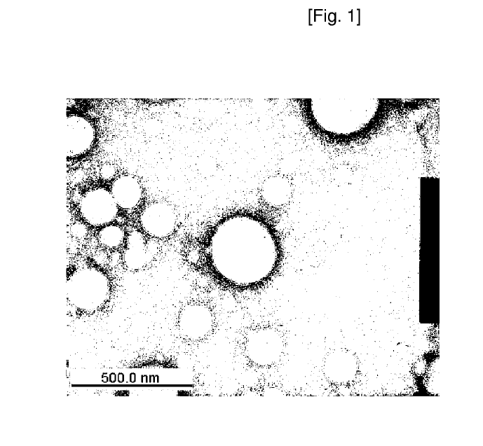

Preparation of Liposome 2.3 mg of {3-{2-[2-(2-methoxy-ethoxy)-ethoxy]-ethylsulfanyl}-propyloxy}12 cucurbituril was dissolved in 0.1 mL of methyl alcohol, and the resultant solution was dried in the air. 6 mL of distilled water was added to the dried product, the temperature of a water bath was controlled to 40° C., and then the product was dispersed in the distilled water for about 30 minutes using sonication. The formation of liposomes having sizes of several tens to 1000 nm was observed using a transmission electron microscope (TEM). The TEM photograph of the liposomes is shown in FIG. 1

### EXAMPLE 2

Preparation of Liposome Having a Surface Modified with Mannose

2.3 mg of {3-{2-[2-(2-methoxy-ethoxy)-ethoxy]-ethylsulfanyl}-propyloxy}12 cucurbituril was dissolved in 0.1 mL of methyl alcohol, and the resultant solution was dried in the air. 6 mL of distilled water was added to the dried product, the temperature of a water bath was controlled to 40° C., and then the product was dispersed in the distilled water for about 30 minutes using sonication to obtain a dispersion of liposomes. 0.5 mg of mannose-spermidine having substitute spermidine at C1 position of mannose was added to the obtained dispersion and then stirred for 1 hour. A residual, non-embedded mannose-spermidine compound was removed by dialysis for 1 day. The formation of liposomes having sizes of several tens to 1000 nm was observed using a TEM.

### EXAMPLE 3

Albumin Encapsulated Liposome

2.3 mg of {3-{2-[2-(2-methoxy-ethoxy)-ethoxy]-ethylsulfanyl}-propyloxy}12 cucurbituril was dissolved in 1 mL of methyl alcohol, and the resultant solution was completely dried. 6 mL of an aqueous solution in which 5 mg of albumin was dissolved was added to the dried product, the temperature of a water bath was controlled to 40□, and then the product was dispersed in the aqueous solution for 30 minutes using sonication. The formation of liposomes having sizes of several tens to 1000 nm was observed using a TEM.

### EXAMPLE 4

Hydrocortisone Encapsulated Liposome

2.3 mg of {3-{2-[2-(2-methoxy-ethoxy)-ethoxy]-ethylsulfanyl}-propyloxy}12 cucurbituril was dissolved in 0.1 mL of methyl alcohol, and the resultant solution was completely dried. About 6 mL of an aqueous solution in which 1 mg of hydrocortisone was dissolved was added to the dried product, the temperature of a water bath was controlled to 40° C., and then the product was dispersed in the aqueous solution for 30 minutes using sonication. The formation of liposomes having sizes of several tens to 1000 nm was observed using a TEM.

### EXAMPLE 5

Insulin Encapsulated Liposome

Liposomes were prepared in the same manner as in Example 4, except that insulin was used instead of hydrocortisone. The formation of liposomes having sizes of several tens to 1000 nm was observed.

### EXAMPLE 6

Calcitonin Encapsulated Liposome

Liposomes were prepared in the same manner as in Example 4, except that 2 mg of calcitonin was used instead of 1 mg of hydrocortisone. The formation of liposomes having sizes of several tens to 1000 nm was observed.

### EXAMPLE 7

Preparation of Albumin Encapsulated Liposome Having Surface Modified with Mannose

2.3 mg of {3-{2-[2-(2-methoxy-ethoxy)-ethoxy]-ethylsulfanyl}-propyloxy}12 cucurbituril was dissolved in 0.1 mL of methyl alcohol, and the resultant solution was dried in the air. 6 mL of an aqueous solution in which 1 mg of albumin was dissolved was added to the dried product, the temperature of a water bath was controlled to 40° C., and then the product was dispersed in the aqueous solution for 30 minutes using sonication to obtain a dispersion of liposomes. 0.5 mg of a mannose-spermidine compound having substitute spermidine at C1 position of mannose was added to the obtained dispersion and then stirred for 1 hour. A residual, non-encapsulated mannose-spermidine compound was removed by dialysis for 1 day. The formation of liposomes having sizes of several tens to 1000 nm was observed using a TEM.

According to the present invention, a liposome formed by self-assembling a cucurbituril derivative of formula 1 above, said liposome encapsulating a drug, said liposome having a surface modified with a targeting compound, and methods of preparing the liposomes are provided.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A liposome formed by self-assembling a cucurbituril derivative having formula 1: 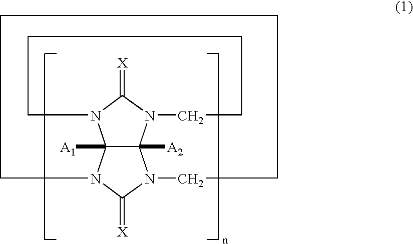
wherein
X is O, S, or NH,
A₁ and A₂ are respectively OR¹ and OR², SR¹ and SR², or NHR¹ and NHR²,
each of R¹ and R² is independently selected from the group consisting of a hydrogen atom, a substituted or unsubstituted C₁-C₃₀ alkyl, a substituted or unsubstituted C₂-C₃₀ alkenyl, a substituted or unsubstituted C₂-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylthiol, a substituted or unsubstituted C₁-C₃₀ alkoxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, and a substituted or unsubstituted C₄-C₂₀ heteroarylalkyl, and
n is an integer from 4 to 20.

2. The liposome of claim 1, wherein a targeting compound is included in a cavity of the cucurbituril derivative composing the liposome such that a targeting moiety of the targeting compound is exposed to the outside of the liposome.

3. The liposome of claim 2, wherein the targeting compound has formula 2:
A-B-T (2)
wherein
A is 1,3-diaminopropyl, 1,4-diaminobutyl, 1,5-diaminopentyl, 1,6-diaminohexyl, sperminyl, spermidinyl, propylamino, butylamino, pentylamino, hexylamino, biologinyl, pyridinyl, ferrocenyl, or amino acid;
B is a hydrogen atom, a substituted or unsubstituted C₁-C₃₀ alkyl, a substituted or unsubstituted C₁-C₃₀ alkenyl, a substituted or unsubstituted C₁-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylsulfanyl, a substituted or unsubstituted C₁-C₃₀ alkyloxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, or a substituted or unsubstituted C₄-C₂₀ heteroarylalkyl; and
T is a saccharide, a polypeptide, a protein, or a gene.

4. The liposome of claim 3, wherein the saccharide is glucose, mannose, or galactose.

5. The liposome of claim 3, wherein the protein is lectin, selectin, or transferrin.

6. The liposome of claim 1, wherein a pharmacologically active substance is encapsulated as a guest molecule in the liposome.

7. The liposome of claim 2, wherein a pharmacologically active substance is encapsulated as a guest molecule in the liposome.

8. The liposome of claim 6, wherein the pharmacologically active substance is an organic compound, a protein, or a gene.

9. The liposome of claim 8, wherein the organic compound is hydrocortisone, prednisolone, spironolactone, testosterone, megesterol acetate, danasole, progesterone, indomethacin, amphotericin B, or a mixture thereof.

10. The liposome of claim 8, wherein the protein is a human growth hormone, a G-CSF (granulocyte colony-stimulating factor), a GM-CSF (granulocyte-macrophage colony-stimulating factor), erythropoietin, a vaccine, an antibody, insulin, glucagon, calcitonin, an ACTH (adrenocorticotropic hormone), somatostatin, somatotropin, somatomedin, parathyroid hormone, thyroid hormone, a hypothalamus secretion, prolactin, endorphin, a VEGF (vascular endothelial growth factor), enkephalin, vasopressin, a nerve growth factor, non-naturally occurring opioid, interferon, asparaginase, alginase, superoxide dismutase, trypsin, chymotrypsin, pepsin, or a mixture thereof.

11. A method of preparing the liposome of claim 1, comprising:
dissolving a cucurbituril derivative having formula 1 in an organic solvent and drying the resultant solution; and
adding water to the dried compound and dispersing the compound, 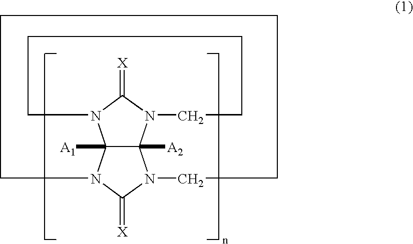
wherein
X is O, S, or NH,
A₁ and A₂ are respectively OR¹ and OR², SR¹ and SR², or NHR¹ and NHR²,
each of R¹ and R² is independently selected from the group consisting of a hydrogen atom, a substituted or unsubstituted C₁-C₃₀ alkyl, a substituted or unsubstituted C₂-C₃₀ alkenyl, a substituted or unsubstituted C₂-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylthiol, a substituted or unsubstituted C₁-C₃₀ alkoxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, and a substituted or unsubstituted C₄-C₂₀ heteroarylalkyl, and
n is an integer from 4 to 20.

12. A method of preparing the liposome of claim 2, comprising:
dissolving a cucurbituril derivative having formula 1 in an organic solvent and drying the resultant solution;
adding water to the dried compound and dispersing the compound;
adding a targeting compound or a solution of the targeting compound to the dispersion and stirring the resultant mixture; and
removing a residual unembedded targeting compound by dialysis, 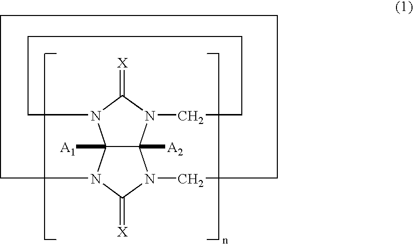
wherein
X is O, S, or NH,
A₁ and A₂ are respectively OR¹ and OR², SR¹ and SR², or NHR¹ and NHR²,
each of R¹ and R² is independently selected from the group consisting of a hydrogen atom, a substituted or unsubstituted C₁-C₃₀ alkyl, a substituted or unsubstituted C₂-C₃₀ alkenyl, a substituted or unsubstituted C₂-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylthiol, a substituted or unsubstituted C₁-C₃₀ alkoxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, and a substituted or unsubstituted C₄-C₂₀ heteroarylalkyl, and
n is an integer from 4 to 20.

13. A method of preparing the liposome of claim 6, comprising:
dissolving a cucurbituril derivative having formula 1 in an organic solvent and drying the resultant solution;
adding an aqueous solution of the pharmacologically active substance to the dried compound and dispersing the compound; and
removing a residual non-encapsulated pharmacologically active substance in the dispersion by dialysis, 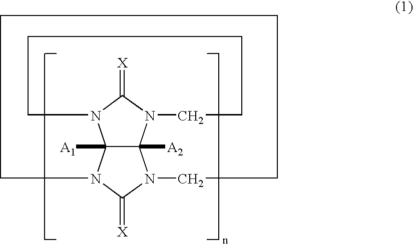
wherein
X is O, S, or NH,
A₁ and A₂ are respectively OR¹ and OR², SR¹ and SR², or NHR¹ and NHR²,
each of R¹ and R² is independently selected from the group consisting of a hydrogen atom, a substituted or unsubstituted C₁-C₃₀ alkyl, a substituted or unsubstituted C₂-C₃₀ alkenyl, a substituted or unsubstituted C₂-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylthiol, a substituted or unsubstituted C₁-C₃₀ alkoxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, and a substituted or unsubstituted C₄-C₂₀ heteroarylalkyl, and
n is an integer from 4 to 20.

14. A method of preparing the liposome of claim 7, comprising:
dissolving a cucurbituril derivative having formula 1 in an organic solvent and drying the resultant solution;
adding an aqueous solution of the pharmacologically active substance to the dried compound and dispersing the compound;
adding a targeting compound or a solution of the targeting compound to the dispersion and stirring the resultant mixture; and
removing a residual non-encapsulated pharmacologically active substance and a residual unembedded targeting compounds by dialysis, 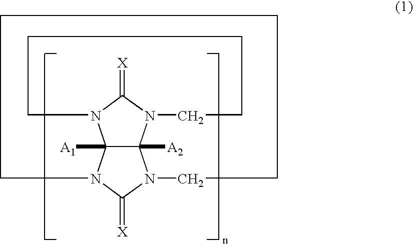
wherein
X is O, S, or NH,
A₁ and A₂ are respectively OR¹ and OR², SR¹ and SR², or NHR¹ and NHR²,
each of R¹ and R² is independently selected from the group consisting of a hydrogen atom, a substituted or unsubstituted C₁-C₃₀ alkyl, a substituted or unsubstituted C₂-C₃₀ alkenyl, a substituted or unsubstituted C₂-C₃₀ alkynyl, a substituted or unsubstituted C₂-C₃₀ carbonylalkyl, a substituted or unsubstituted C₁-C₃₀ thioalkyl, a substituted or unsubstituted C₁-C₃₀ alkylthiol, a substituted or unsubstituted C₁-C₃₀ alkoxy, a substituted or unsubstituted C₁-C₃₀ hydroxyalkyl, a substituted or unsubstituted C₁-C₃₀ alkylsilyl, a substituted or unsubstituted C₁-C₃₀ aminoalkyl, a substituted or unsubstituted C₁-C₃₀ aminoalkylthioalkyl, a substituted or unsubstituted C₅-C₃₀ cycloalkyl, a substituted or unsubstituted C₂-C₃₀ heterocycloalkyl, a substituted or unsubstituted C₆-C₃₀ aryl, a substituted or unsubstituted C₆-C₂₀ arylalkyl, a substituted or unsubstituted C₄-C₃₀ heteroaryl, and a substituted or unsubstituted C₄-C₂₀ heteroarylalkyl, and
n is an integer from 4 to 20.

15. The method of claim 11, wherein the organic solvent is chloroform, methyl alcohol, dimethylsulfoxide, dichloromethane, dimethylformamide, tetrahydrofuran, or a mixture thereof.

16. The method of claim 11, wherein the dispersing is performed by sonication with a sonicator.
